(19) United States
(12) Patent Application Publication
(10) Pub. No.: US 2007/0233233 A1
(43) Pub. Date: Oct. 4, 2007

(54) Tethered expansion columns for controlled stent expansion

(75) Inventors: Jim McGovern, Eden Prairie, MN (US); Daniel Gregorich, St. Louis Park, MN (US); Liza Davis, St. Michael, MN (US); Michael P. Meyer, Richfield, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC, Maple Grove, MN (US)

(21) Appl. No.: 11/395,743

(22) Filed: Mar. 31, 2006

(51) Int. Cl. A61F 2/06

(52) U.S. Cl. 623/1.15

(57) ABSTRACT

A tethered stent made up of interconnected struts which together form expansion columns. At least some of the struts are connected to each other by tethers. When the stent expands, the tethers pull on the interconnected struts and limit how far apart from each other the struts can move when expanding. This limitation allows for greater control over what overall shape the expanded stent will assume and how particular portions of the stent will deploy. The tethers also add density to the stent and increases surface area which leads to better stent scaffolding and greater effectiveness of drug coatings.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

### Field of the Invention

In some embodiments this invention relates to implantable medical devices, their manufacture, and methods of use. Some embodiments are directed to delivery systems, such as catheter systems of all types, which are utilized in the delivery of such devices.

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

All US patents and applications and all other published documents mentioned anywhere in this application are incorporated herein by reference in their entirety.

### BRIEF SUMMARY OF THE INVENTION

This invention contemplates a number of embodiments where any one, any combination of some, or all of the embodiments can be incorporated into a stent and/or a stent delivery system and/or a method of use. In the context of these embodiments, the term telescoping means to extend away from a stent wall in a direction different from that of the longitudinal axis of a stent. Telescoping includes but is not limited each or any combination of: extending along a linear, varied, or curved path; extending at an oblique angle from the longitudinal axis of the stent; as well as extending along a path parallel to the longitudinal axis of the stent.

At least one possible embodiment of the inventive concept is directed towards a stent having an unexpanded state and an expanded state. The stent comprises a stent body. The stent body comprises at least one expansion column. The expansion column comprises a plurality of interconnected adjacent strut members and at least one tether. Each strut member has a length extending between two ends. Each strut member is engaged at one end to one adjacent strut member. Each of the plurality of strut members has an untethered unexpanded distance in the unexpanded state relative to each other and in the expanded state has an untethered expanded distance relative to each other. The untethered expanded distance is different than the untethered unexpanded distance. The at least one tether is engaged to and extends between a position on each of the struts of at least one pair of the plurality of strut members. In the unexpanded state the at least one tether has a less taut configuration. The at least one pair of struts has an unexpanded tethered distance between the two struts members of the pair. In the expanded state the at least one tether has a more taut configuration and the at least one pair of struts has an expanded tethered distance between the two strut members of the pair. The untethered expanded distance is not less than the expanded tethered distance.

At least one possible embodiment of the inventive concept is directed towards a stent in which the at least one pair of strut members is engaged to the at least one tether are adjacent strut members.

At least one possible embodiment of the inventive concept is directed towards a stent further comprising at least one untethered pair defining two adjacent strut members which are not directly engaged to each other by a tether extending therebetween. In the expanded state, the distance between the two adjacent strut members of the untethered pair is greater than the expanded tethered distance.

At least one possible embodiment of the inventive concept is directed towards a stent in which the at least one expansion column comprises a plurality of undulating adjacent strut members. Each strut member has two ends and each strut is interconnected to at least one adjacent strut at a position located by at least one of the two ends.

At least one possible embodiment of the inventive concept is directed towards a stent in which there are a plurality of adjacent expansion columns each expansion column comprising a plurality of cells defining the space between the strut members of the expansion columns. Each of the cells of one expansion column are adjacent to a corresponding cell in the adjacent expansion column. At least two adjacent cells on adjacent expansion columns have tethers connecting the struts of the two adjacent cells.

At least one possible embodiment of the inventive concept is directed towards a stent in which the stent further comprises a generally tubular body and a side branch assembly. The generally tubular stent body defines a first circumferential plane. The stent body defines a first lumen with a first longitudinal axis extending therethrough. The stent body further defines at least one side opening. The at least one side opening is in fluid communication with the first lumen. The side branch assembly is engaged to the stent body adjacent to the side opening. In the unexpanded state, the side branch assembly is positioned substantially within the first circumferential plane. In the expanded state at least a portion of the side branch assembly extends above the first circumferential plane and defines a second lumen with a second longitudinal axis extending therethrough. The second longitudinal axis forms a default angle which defines the oblique angle formed between the second longitudinal axis and the first longitudinal axis.

At least one possible embodiment of the inventive concept is directed towards a stent in which at least one strut member is adjacent to the side branch assembly and has a tether connecting it to another strut member.

At least one possible embodiment of the inventive concept is directed towards a stent in which the tether exerts a tensional force which pulls the side branch assembly into a configuration in which the second longitudinal axis moves into a further acute orientation relative to the first longitudinal axis than in the default angle.

At least one possible embodiment of the inventive concept is directed towards a stent in which in the expanded state at least a portion of the stent body comprises a curved cylindrical region. The curved cylindrical region of the stent body defines a bend. The bend comprises an outside of a bend located on the concave side of the curved cylindrical region and an inside of a bend located on the convex side of the curved cylindrical region.

At least one possible embodiment of the inventive concept is directed towards a stent in which the at least one tether is located on the outside of the bend.

At least one possible embodiment of the inventive concept is directed towards a stent in which a plurality of struts are engaged to tethers of progressively increasing length relative to their proximity to the outside of the bend.

At least one possible embodiment of the inventive concept is directed towards a stent in which a plurality of expansion columns posses a plurality of tethers connecting the strut members of a plurality of cells.

At least one possible embodiment of the inventive concept is directed towards a stent in which at least two of the tethered struts in an expansion column are not adjacent to each other.

At least one possible embodiment of the inventive concept is directed towards a stent in which in the unexpanded state, at least one tether has a nonlinear shape.

At least one possible embodiment of the inventive concept is directed towards a stent in which in the unexpanded state, at least one tether has one shape selected from the list consisting of: curved, rounded, angled, looped, and omega shaped.

At least one possible embodiment of the inventive concept is directed towards a stent in which the stent body and the at least one tether are both cut out of a common tube of material.

At least one possible embodiment of the inventive concept is directed towards a stent in which at least a portion of the cut is cut with a laser.

At least one possible embodiment of the inventive concept is directed towards a stent in which the stent body is created out of a primary material source and the at least one tether is constructed out of a secondary material source, and the at least one tether.

At least one possible embodiment of the inventive concept is directed towards a stent in which the secondary material is one selected from the list consisting of: drug coated elastic bands, PTFE, and suture material.

At least one possible embodiment of the inventive concept is directed towards a stent having an expanded state and an unexpanded state. The stent comprises a stent body. The stent body comprises at least one expansion column and has a length. The expansion column comprises at least three interconnected strut members and at least one tether. Of the at least three interconnected strut members, at least two of the strut members are non-adjacent to each other. The at least three strut members expand along at least a portion of the expansion column. The at least one tether is engaged to at least two of the at least two non-adjacent strut members. The distance between at the least two non-adjacent struts in the unexpanded state is different than the distance between the at least two non-adjacent struts in the expanded state.

### BRIEF DESCRIPTION OF THE SEVERAL VIEWS OF THE DRAWINGS

The invention is best understood from the following detailed description when read in connection with accompanying drawings, in which:

FIG. 1A is a lateral view of a portion of an unexpanded stent expansion column with tether linked struts.

FIG. 1B is a lateral view of a portion of an expanded stent expansion column with tether linked struts.

FIG. 1C is a lateral view of an unexpanded stent with tether linked struts.

FIG. 2A is a lateral view of a portion of an unexpanded stent expansion column with tether linked struts on a bifurcated stent.

FIG. 2B is a lateral view of a portion of an expanded stent expansion column with tether linked struts on a bifurcated stent.

FIG. 3A is a PRIOR ART view of a portion of an expanded stent expansion column on a bent stent.

FIG. 3B is a lateral view of a portion of an expanded stent expansion column with tether linked struts on a bent stent.

FIG. 4A is a lateral view of a portion of an unexpanded stent expansion column with a tether linked strut section.

FIG. 4B is a lateral view of a portion of an expanded stent expansion column with a tether linked strut section.

FIG. 5A is a lateral view of a portion of an unexpanded stent expansion column delineating the extent of columnar expansion.

FIG. 5B is a lateral view of a portion of an expanded stent expansion column delineating the extent of columnar expansion.

FIG. 6A is a lateral view of a portion of an unexpanded stent expansion column with specifically oriented strut linking tethers.

FIG. 6B is a lateral view of a portion of an expanded stent expansion column with specifically oriented strut linking tethers.

FIG. 7A is a lateral view of a portion of an expanded stent expansion column with a first group of differently shaped tethers.

FIG. 7B is a lateral view of a portion of an expanded stent expansion column with a second group of differently shaped tethers.

FIG. 7C is a lateral view of a portion of an expanded stent expansion column with a third group of differently shaped tethers.

### DETAILED DESCRIPTION OF THE INVENTION

The invention will next be illustrated with reference to the figures wherein the same numbers indicate similar elements in all figures. Such figures are intended to be illustrative rather than limiting and are included herewith to facilitate the explanation of the apparatus of the present invention.

For the purposes of this disclosure, like reference numerals in the figures shall refer to like features unless otherwise indicated.

Depicted in the figures are various aspects of the invention. Elements depicted in one figure may be combined with, or substituted for, elements depicted in another figure as desired.

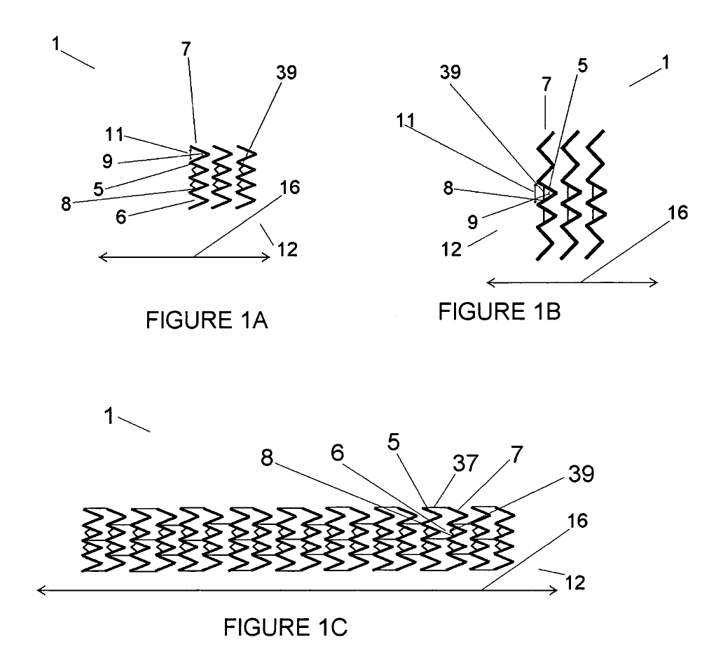

In some embodiments of the inventive concept, a stent utilizes a plurality of circumferential expansion columns 7 which expand to provide support to the target body vessel it is implanted within. FIG. 1A shows a region of the body of an unexpanded stent 1 which comprises a plurality of expansion columns 7 each column comprising a plurality of adjacent strut members 5 which define the outer walls of a cell 6. As illustrated in FIG. 1B, as the stent expands, the distance 11 between the strut members 5 changes. Spanning between at least two strut members 5 are at least one tether 39. The tether 39 has a maximum expansion length which is less than or equal to the maximum inter-strut distance 11 that can result from stent expansion. The presence of the tether 39 limits the inter-strut distance 11 resulting from stent expansion to the maximum expansion length of the tether 39.

The addition of tethers to the columns can provide a number of advantages which improve the utility of the stent 1. Tethers 39 can provide the ability to more precisely control the expansion of selected regions of the stent body 1. Controlling the amount of distance 11 between strut members 5 can also affect the density characteristics of particular regions of the stent as well as provide greater strut uniformity in the tethered region. In addition, controlling the expansion resulting inter-strut member distance can increase the stent body surface area to body vessel wall area ratio and can force the expanded stent to assume particular geometry characteristics in non tethered regions of the stent. The tethers can be of various sizes, shapes, and orientations to accommodate specific deployment characteristics.

In the embodiment shown in FIGS. 1A and 1B, a plurality of adjacent undulating type expansion columns 7 having adjacent strut members 5 with cells 6 between the strut members is shown. In every column 7 there are uniformly dimensioned tethers 5 positioned between engagement points 8 on the strut members 5 which span each cell 6. m

Although FIGS. 1A and 1B illustrate a stent having unconnected columns, the inventive concept is equally applicable to a stent in which the columns or strut members are interconnected or otherwise engaged to each other by connector members. FIG. 1C illustrates the inventive concept on an unexpanded stent in which the expansion columns 7 are interconnected by connectors 37 as well as by tethers 39.

In addition, this inventive concept is not limited to undulating expansion columns and can be applied to any stent geometry. Similarly the positioning of the tethers can be done in various locations resulting in a variety of expansion geometries depending on the specific applications. For example a stent or portion thereof may include any number of tethered expansion columns 7 such as are described. In some embodiments of the invention, the expansion columns 7 of the entire stent may include tethers 39 between some or all of the adjacent strut member 5 pairs. In at least one embodiment, a stent comprises tethered expansion columns 7 in the region of the stent adjacent to a side branch opening, such as in a stent for implantation at a vessel bifurcation. The tethers can also be of similar or different lengths, thicknesses, widths, materials, overall shapes, and can induce similar or different resistive tension to stent expansion.

There are a number of ways in which the tethered expansion columns 7 can be constructed. Tether lengths can be cut or laser cut out of the same material as the expansion column 7. In at least one possible embodiment of this inventive concept, the stent and the tether 39 both are laser cut or carved from a single tube of material. The tethers can also be made out of a secondary material that can be attached or otherwise engaged to the body of the stent 1. In at least one possible embodiment of this inventive concept, the tether is constructed out of a secondary material including but not limited to drug coated elastic bands, PTFE, or at least one suture material. In at least one possible embodiment, the tether in engaged to the stent body after the stent body or at least some of its other components or integrated features are assembled, carved, or laser cut.

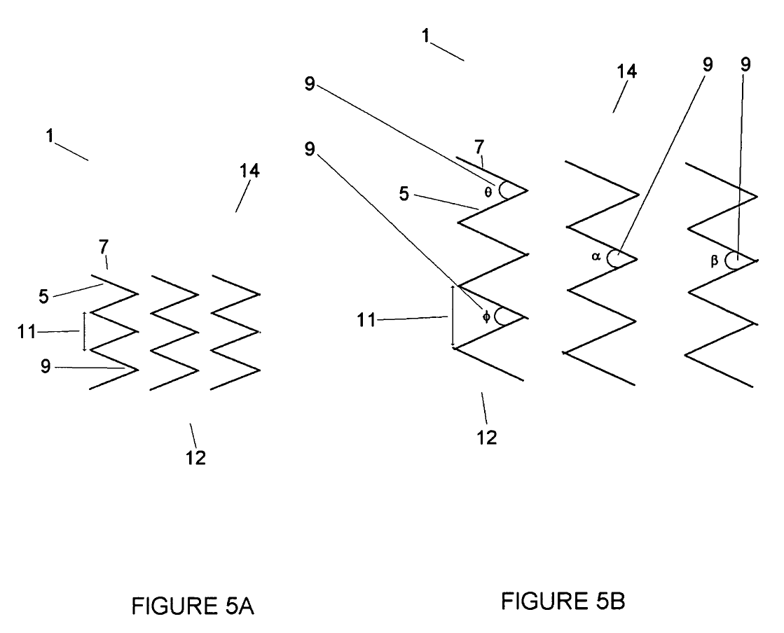

Some details of this inventive concept can be better understood with reference to FIG. 5B. FIG. 5B shows that in the case of undulating expansion columns 7, the cells 6 can be said to have expansion angles 9, for example angles α, β, θ, and Φ. In the absence of a tether or some other intervening effect, in the expanded state the cells 6 will expand uniformly resulting in the α=β=θ=Φ relationship. If however a given cell 6 has a tether connecting the adjacent bordering strut members 5 and the maximum length of the taut tether is less than the maximum possible distance 11 formed between the engagement points of the tether when the expansion angle 9 has its maximum possible value, then the values of the various expansion angles 9 can be controlled and can be differentiated. The presence of tethers between engagement points can potentially result in some or all of α, β, θ, and Φ being unequal. In embodiments of stent bodies which do not have undulating expansion members, the tethers can be said to change the resulting distance 11 between engagement points 8 of adjacent struts in the stent body 1.

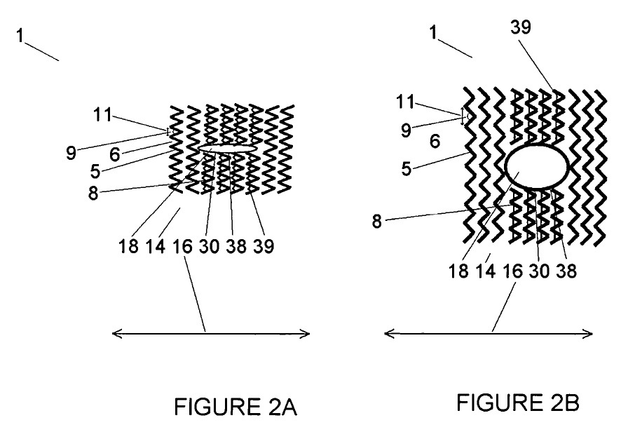

In at least one possible embodiment of the inventive concept, as illustrated in FIGS. 2A and 2B, the stent 1 having tethered expansion columns 7 is a bifurcated stent. The main stent body of the bifurcated stent can be said to extend along the length of a first longitudinal axis 16. The bifurcated stent has a side branch assembly 30 adjacent to a side opening 18 in the main body of the stent 1. In the unexpanded state, the side branch assembly 30 lies generally along a circumferential plane 12 of the first lumen 14 defined by the main body 1 of the stent. In the expanded state, at least a part of the side branch assembly 30 extends outside of the circumferential plane 12 of the first lumen and defines a second lumen in fluid communication with the first lumen. The second lumen extends into a branch of the body vessel into which the stent is implanted. The second lumen can be said to extend about a second longitudinal axis which defines the deployment vector of the second fluid lumen and which forms an oblique angle relative to the first longitudinal axis.

In addition to all of the improved characteristics of the stent illustrated in FIGS. 1A and 1B, the tethered bifurcated stent of FIGS. 2A and 2B has characteristics of particular significance for a bifurcated stent. When expanding, the tethers can provide lateral or radial force on to the side branch assembly 30 which can push or pull at the ostium 38 of the side branch assembly 30 and define a wider or narrower second lumen. In addition, the tethers 7 or the tether engaged strut members 5 can be engaged to the side branch assembly 30 at a point above and or below the side branch assembly 30 relative to the circumferential plane 12 which can modify the angle second longitudinal axis of relative to the first longitudinal axis 14.

Although stent expansion generally increases the distance 11 between adjacent strut members 5, in some instances the stent expansion can cause localized compression which would move some adjacent strut members 5 closer together. Localized strut compression in one region of the stent can in turn cause hyper expansion which can result in excessive inter-strut distances 11 in other regions of the stent.

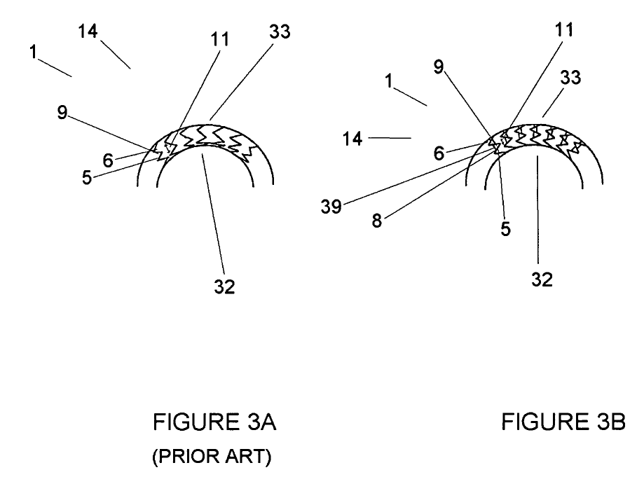

One possible embodiment of the inventive concept utilizing tethered strut members to regulate localized strut member compression and or hyper expansion is illustrated in novel FIG. 3B which can be contrasted with PRIOR ART FIG. 3A. A bent stent can be bent when unexpanded, can be substantially straight when unexpanded and assume a bent configuration when expanded, and/or can assume a bent configuration as a result of the anatomical configuration of a vessel(s) or other body lumen(s) through with the stent is advanced and/or deployed. PRIOR ART FIG. 3A shows that typically in a bent stent 1, an expansion column 7 tends to become more compressed at the bend concavity 32 and more diffuse at the bend convexity 33. A more uniform scaffolding design can be achieved by the presence of tethers 39 between adjacent struts 5 on regions of the expansion column 7 closer to the bend convexity 33. These tethers 39 limit the extent to which the struts 5 at the convexity 33 can separate from each other resulting in a separation substantially uniform with those on the concavity 32. In at least one possible embodiment of this inventive concept the uniform scaffolding of the bent stent is used to improve the distribution of a drug coating on the stent.

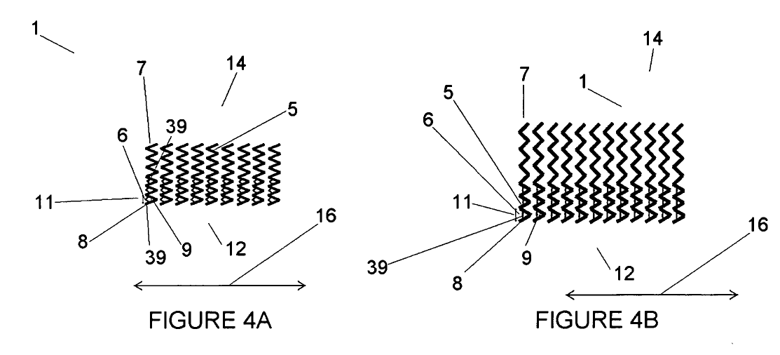

In at least one possible embodiment of the inventive concept, as illustrated in FIGS. 4A and 4B, there is shown a portion of a stent body 1 in which a particular region of the stent body has tethers 39 spanning between the strut members 5 of expansion columns 7. The distribution of multiple tethers 39 across portions of multiple expansion columns 7 can cause regions of the expansion column 7 to expand unevenly. This in turn can cause the expanded stent to assume a shape which defines a lumen having a configuration significantly deviant from standard tubular shaped stent lumens. Such circumferentially non-uniform shaped expanded stent lumens can be used to implant a stent into an eccentrically shaped body vessel, to enable specific geometry based scaffolding effects, or to regulate intra-luminal fluidic flux.

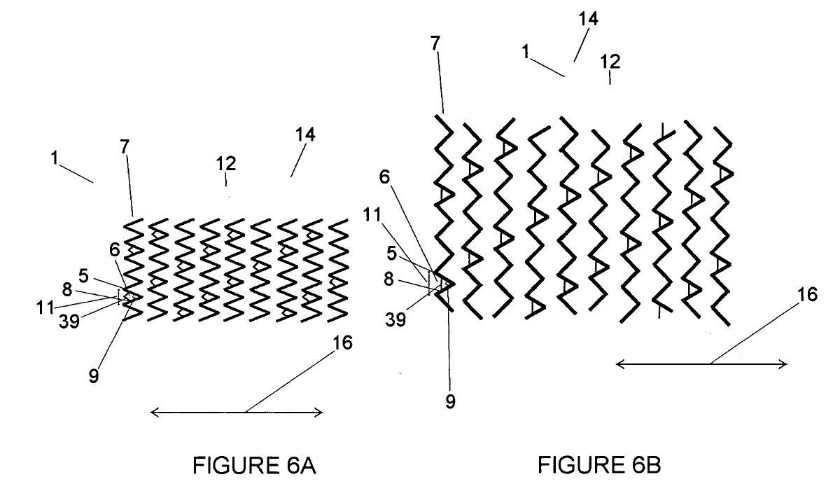

Referring now to FIGS. 6A and 6B there are shown regions of stent bodies 1 having tethers 39 spanning between the engagement points 8 of adjacent strut members 5 in expansion columns 7. In this embodiment of the inventive concept, at least some of the tethered cells 6 of one expansion column 7 are not adjacent to the tethered cells 6 of the adjacent expansion column 7. Non-adjacent tethering of expansion columns can produce specific stent geometric configurations. It can also result in particular density characteristics at specific locations on the stent body 1.

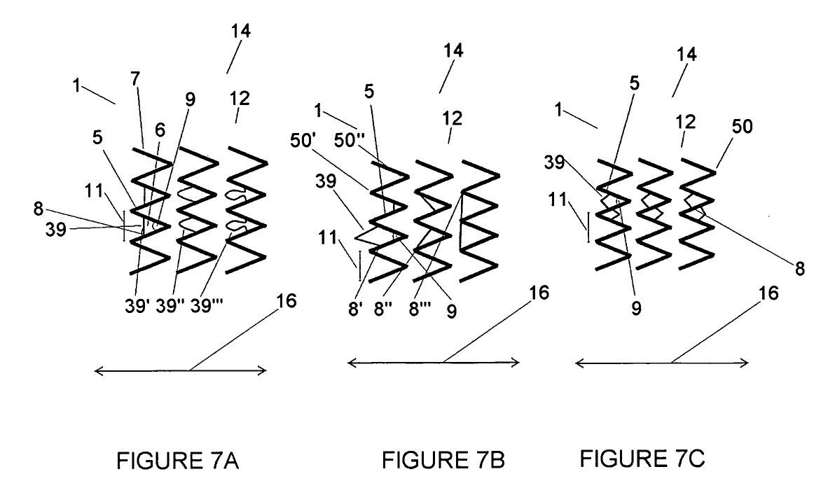

Referring to FIG. 7A there are shown tethered stent expansion columns having tethers 39 with three distinct shape types, straight tethers 39′, curved tethers 39″, and omega shaped tethers 39′″. The tethers 39 can be of a multitude of possible shapes including but not limited to the types shown FIG. 7A, rounded, compressed, angular, or any shape known in the art. Similarly there is no limit to the number of types of tethers that can connect strut members 5. The tether shapes can allow the expansion of the expansion columns to be highly limited or can allow the maximum inter-strut distance 11 to be achieved. In addition, tethers that have no effect on the maximum resulting distance 11 between the struts 5 can connect the struts 5. Besides for their effect on the expansion of cells, the tethers can add surface area, scaffolding strength, and density to the stent body.

Referring to FIG. 7B there are shown tethered stent expansion columns having tethers 39 with different engagement points 8. FIG. 7B shows a peak engagement point 8″, and a central engagement point 8′. The engagement points 8 can be located at any point on the strut including but not limited to at the peaks 50 or at a point along the struts 5 between the peaks. For the purposes of this application, the term “peak” refers to the most distal and/or the most proximal point of a strut 5. Adjacent struts 5 can have the same or different engagement points 8. In addition, there can be non-adjacent engagement points 8′″ where the tether 39 can span between non-adjacent cells 6 and can extend past more than one cell 6.

Referring to FIG. 7C there are shown tethered stent expansion columns having tethers 39 with more than one tether 39 per strut 5. Struts with more than one tether 39 can have their respective engagement points 8 at the same positions on the strut 5 or at positions closer to the peaks 50. Similarly, struts 5 with more than one tether 39 can have the multiple tethers 39 span different cells 6. In addition, the tethers 39 can be located on any or all sides of the cells including but not limited to the distal and proximal sides of the cells.

In some embodiments the stent, its delivery system, or other portion of an assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

This completes the description of the preferred and alternate embodiments of the invention. The above disclosure is intended to be illustrative and not exhaustive. This description will suggest many variations and alternatives to one of ordinary skill in this art. The various elements shown in the individual figures and described above may be combined, substituted, or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claims below.

1. A stent having an expanded state and an unexpanded state, the stent comprising at least one expansion column, the at least one expansion column comprising a plurality of strut members each having two ends and a length extending therebetween, each strut member sharing a common end with an adjacent strut member to define a plurality of interconnected strut pairs, each strut pair defining a predetermined distance between a position on each of the strut members, the distance being greater in the expanded state than in the unexpanded state, at least one strut pair having a tether engaged to and extending between the position on each of the interconnected strut members, the tether having a length, the length of the tether being less than the distance in the expanded state.

2. The stent of claim 1 wherein the stent comprises a plurality of longitudinally adjacent expansion columns, in the expanded state the at least one expansion column has a first diameter and at least one adjacent expansion column has a second diameter, the first diameter being less than the second diameter.

3. The stent of claim 1 in which in the unexpanded state, at least one tether has a nonlinear shape.

4. The stent of claim 1 in which in the unexpanded state, at least one tether has one shape selected from the list consisting of: curved, rounded, angled, looped, and omega shaped.

5. The stent of claim 1 in which the stent body and the at least one tether are both cut out of a common tube of material.

6. The stent of claim 5 in which the common tube of material is cut by a laser.

7. The stent of claim 1 in which the stent body is created out of a primary material source and the at least one tether is constructed out of a secondary material source.

8. The stent of claim 7 in which the secondary material source is one selected from the list consisting of: drug coated elastic bands, PTFE, and suture material.

9. The stent of claim 1 in which the stent further comprises: a generally tubular stent body defining a first circumferential plane, the stent body defining a first lumen with a first longitudinal axis extending therethrough, the body further defining at least one side opening, the at least one side opening in fluid communication with the first lumen;
a side branch assembly, the side branch assembly engaged to the stent body adjacent to the side opening;
in the unexpanded state, the side branch assembly being positioned substantially within the first circumferential plane, in the expanded state at least a portion of the side branch assembly extending above the first circumferential plane and defining a second lumen with a second longitudinal axis extending therethrough, the second longitudinal axis forming a default angle defining the oblique angle formed between the second longitudinal axis and the first longitudinal axis.

10. The stent of claim 9 in which at least one strut member is adjacent to the side branch assembly and has a tether connecting it to another strut member.

11. The stent of claim 9 in which the tether exerts a tensional force which pulls the side branch assembly into a configuration in which the second longitudinal axis moves into a further acute orientation relative to the first longitudinal axis than in the default angle.

12. A stent having an expanded state and an unexpanded state,
in the expanded state at least a portion of the stent body assuming a curved cylindrical shape, the curved cylindrically shaped portion of the stent body defining a bend, the bend having a concave side and a convex side
the stent comprising at least one expansion column, the expansion column comprising a plurality of strut members each having two ends and a length extending therebetween, each strut member sharing a common end with an adjacent strut member to define a plurality of interconnected strut pairs, each strut pair defining a predetermined distance between a position on each of the strut members,
at least one strut pair positioned on the concave side of the bend having a tether engaged to and extending between the position on each of the interconnected strut members.

13. The stent of claim 12 in which a plurality of strut pairs having tethers engaged to and extending between the position on each of the interconnected strut members, the tethers having progressively increasing lengths relative to their proximity to the concave side of the bend.

14. The stent of claim 12 in which at least one tether engaged to a strut member located on the concave side of the bend prevents the distance between a strut pair on the concave side of the bend from being substantially greater than the distance between the strut members of a strut pair on the convex side of the bend.

15. The stent of claim 12 in which the predetermined distance of a plurality of strut pairs on the concave side of the bend are substantially equal to the predetermined distance of a plurality of strut pairs on the concave side of the bend.

16. A stent having an expanded state and an unexpanded state, the stent comprising:
a stent body,
the stent body comprising at least one expansion column,
the at least one expansion column comprising a plurality of interconnected strut members each strut member having a length extending between two ends;
each of the plurality of strut members has an untethered unexpanded distance in the unexpanded state relative to each other and in the expanded state each strut member has an untethered expanded distance relative to each other, the untethered expanded distance being different than the untethered unexpanded distance;
at least one tether is engaged to and extends between a position on a pair of strut members defining a tethered pair of struts, in the unexpanded state the at least one tether has a less taut configuration and the strut members of the at least one tethered pair of struts has an unexpanded tethered distance therebetween,
in the expanded state the at least one tether having a more taut configuration and the at least one tethered pair of struts having an expanded tethered distance therebetween,
the expanded untethered distance being not less than the expanded tethered distance.

17. The stent of claim 16 in which at least one tether is engaged to two strut members positioned on different expansion columns.

18. The stent of claim 16 in which at least one tether is engaged to two strut members who are not adjacent to each other.

19. The stent of claim 16 in which at least two of the tethered struts in an expansion column are not adjacent to each other.

20. The stent of claim 16 in which a strut member is engaged to more than one tether.
